(19) United States
(12) Patent Application Publication
(10) Pub. No.: US 2020/0323559 A1
(43) Pub. Date: Oct. 15, 2020

(54) MIS Access Port and Methods of Using

(71) Applicant: SpineCraft, LLC, Westmont, IL (US)

(72) Inventors: James J. Skinner, Lake Forest, IL (US); Steven E. Mather, Hinsdale, IL (US); Wagdy W. Asaad, Burr Ridge, IL (US); Alan W. Cannon, Lakeport, CA (US)

(21) Appl. No.: 16/843,565

(22) Filed: Apr. 8, 2020

Related U.S. Application Data
(60) Provisional application No. 62/831,357, filed on Apr. 9, 2019.

(51) Int. Cl. A61B 17/34; A61B 90/30; A61B 17/70

(52) U.S. Cl. CPC A61B 17/3462; A61B 17/3423; A61B 2017/00407; A61B 17/7074; A61B 90/30

(57) ABSTRACT

An access port for minimally invasive surgery includes an elongate, tubular main body portion, a slot extending over a length of the main body portion in a lengthwise direction, and a flap which covers the slot in a closed configuration, while exposing the slot when in an open configuration. The slot extends through a wall of the main body portion, from an outside surface of the main body portion to an inside surface of the main body portion.

### CROSS-REFERENCE

This application claims the benefit of U.S. Provisional Application No. 62/831,357, filed Apr. 9, 2019, which application is incorporated herein, in its entirety, by reference thereto.

### FIELD OF THE INVENTION

The present invention relates to minimally-invasive surgery. More particularly the prevent invention relates to minimally-invasive spinal surgery.

### BACKGROUND OF THE INVENTION

Spinal fusion procedures may involve the remove of a herniated disk and cleaning all of the debris out of the disk space prior to introducing an implant such as a cage or the like and grafting material. To perform such procedures as minimally-invasive surgery (MIS), it can be difficult, if not impossible to remove all of the fragments of the disk or any other debris that might impede the proper insertion and placement of one or more implants and, optionally grafting material. One of the major causes of this difficulty is that access ports or other tubes used to allow the surgeon to access the surgical site place significant restrictions on the mobility of the instruments being used by the surgeon to remove the disk, disk fragments and debris.

U.S. Patent Application Publication No. 2014/0243604 provides a surgical access tube that is provided with oppositely positioned weakened distal portions that can be removed to afford lateral intrusions of the spinous process and facet, respectively, to allow placement of the access tube over the spinous process and facet during a surgical procedure. The break-away, weakened distal portions simplify the fitting of the access tube, making it easier to use than previous tubes that the surgeons had previously used after selectively resecting some portion of the distal end of the tube not having the weakened sections. A weakened proximal portion may also be provided to improve a range of angles for surgical instruments working through the access tube. The weakened proximal section is limited to a height of about 15-20 mm and weakened sections converge from a wider base opening (either at the distal end or proximal end of the access tube) to an arc base, presumably to avoid crack propagation and maintain rigidity of the access tube. Due to the limited height and sweep angle defined by the weakened proximal section, angulation of instruments is limited to only about 25 degrees, possibly up to 30 degrees. Further, the tube must be installed in a predetermined orientation relative to the spinous process and facet and therefore does not allow the tube to be rotated about its longitudinal axis, thereby further limiting the ability to angulate instruments inserted therethrough. It would be desirable to provide solutions that would allow a greater range of angulation of instruments used in an access tube.

U.S. Pat. No. 7,594,888 discloses expandable ports that can be used in minimally invasive surgery. The expandable ports are typically enclosed along the lengths thereof, even after expansion, but some embodiments, such as in FIGS. 17, 18, 29 and 23 result in proximal and distal gaps at the location of expansion. Such gaps are merely the result of the mechanism used to expand the tube. Further, these embodiments retain an impediment such as a mechanism intermediate the proximal and distal gaps which would limit angulation much in the same way described above that angulation is limited in U.S. Patent Application Publication No. 2014/0243604.

U.S. Patent Application Publication No. 2016/0270816 provides an access port for minimally invasive surgery that includes an elongate, tubular main body portion, a connector configured to fix at least a portion of the access port to a stationary object; and a slot extending over a length of the main body portion in a lengthwise direction.

There is a need for improved products and methods that allow greater mobility of surgical instruments during MIS surgery, particularly MIS spine surgery.

There is a need for improved products and methods that provide greater, more consistent visibility during MIS surgery, eliminating or significantly reducing shadows and “dead spots” in the visibility field.

There is a need for improved products and methods that, in addition to allowing greater mobility of surgical instruments during MIS surgery, at the same time provide greater protection to the tissues surrounding such products.

There is a need for improved products and methods that, in addition to allowing greater mobility of surgical instruments during MIS surgery, discourage or eliminate tissue creep into such products.

### SUMMARY OF THE INVENTION

According to one aspect of the present invention, an access port configured and dimensioned for use in minimally invasive surgery includes; an elongate, tubular main body portion having a first proximal end portion and a first distal end portion, the distal end portion being configured to be inserted into a patient and at least a portion of the proximal end portion being configured to remain outside of the patient during use; a slot extending over a length of the main body portion in a lengthwise direction; and a flap having a length at least as great as a length of the slot, the flap having a second proximal end portion and a second distal end portion; wherein the flap is pivotally joined to the main body portion by the second distal end portion being pivotally joined to the first distal end portion; wherein the slot has a length and a width and extends through a wall of the main body portion, from an outside surface of the main body portion to an inside surface of the main body portion; wherein, in a closed configuration, the flap closes off the slot; and wherein, upon rotating the flap away from the main body portion to an open configuration, the slot is exposed.

In at least one embodiment, the flap is configured to be stably positioned at any angle relative to a longitudinal axis of the main body portion, between zero degrees and a maximum degree of angulation achievable.

In at least one embodiment, the flap is pivotally mounted to the main body portion via a torque and positioning hinge.

In at least one embodiment, the access port further includes an angulation control mechanism configured to be actuated to drive the flap in a controlled manner to open or close the flap position and maintain an angulation of the flap relative to a longitudinal axis of the main body portion at any angle between zero degrees and a maximum angle of the angulation achievable.

In at least one embodiment, the angulation control mechanism comprises a ratchet mechanism.

In at least one embodiment, the access port further includes skirts interconnecting sides of the flap with edges of walls of the main body that define the slot.

In at least one embodiment, the access port further includes a connector extending from the first proximal end portion, the connector being configured to facilitate fixing the access port to a stationary object.

In at least one embodiment, the access port further includes a port extending through a wall of the first proximal end portion, the port being configured to allow a portion of a lighting instrument to be inserted therethrough.

In at least one embodiment, the access port further includes a light cable installed through the port.

In at least one embodiment, when in the open configuration, the slot allows an instrument inserted into the main body portion to be angled relative to a longitudinal axis of the main body portion, by passing at least a portion of the instrument through the slot.

In at least one embodiment, the slot extends nearly a full length of the main body portion, but is closed off at a distal end of the main body portion where the flap is joined to the main body portion.

In at least one embodiment, a width of the slot is substantially constant over an entirety of a length of the slot.

In at least one embodiment, the slot is tapered such that the slot has a first width along a proximal portion thereof and a second width along a distal portion thereof, with an intermediate portion that tapers from the first width to the second width, wherein the first width is greater than the second width, wherein the first width is constant over an entire length of the proximal portion and the second width is constant over an entire length of the second portion.

In at least one embodiment, the first distal end portion of the main body portion is tapered.

In at least one embodiment, the second distal end portion of the flap is tapered to conform to the tapered, first distal end portion of the main body portion.

In at least one embodiment, the access port further includes at least one longitudinally extending cutout in an inner wall of the main body portion; and at least one light strip mounted in the at least one longitudinally extending cutout, respectively.

In at least one embodiment, the at least one light strip is flush with the inner wall of the main body portion.

In at least one embodiment, the access port is provided in the open configuration, in combination with an instrument extending through the access port, wherein a distal end of the instrument extends distally of a distal end of the access port and a shaft of the instrument extends through the slot, to provide increased range of motion of the distal end of the instrument.

In at least one embodiment, the access port is provided in the closed configuration, in combination with an instrument extending through the access port, wherein a distal end of the instrument extends distally of a distal end of the access port and a shaft of the instrument is prevented from extending through the slot by the flap.

In at least one embodiment, the access port is configured and dimensioned for use in minimally invasive spine surgery.

In at least one embodiment, the access port includes a light guide assembly installed in the first proximal end portion; wherein the light guide assembly comprises a fiber optic cable having optical fibers, the optical fibers being fanned out over an inner circumference of the first proximal end portion to provide even lighting throughout a tubular opening of the tubular main body portion and a surgical target targeted by a distal end opening of the tubular opening.

In at least one embodiment, the light guide assembly further comprises a split compression ring, the optical fibers being adhered to the split compression ring and terminating at or proximal of a distal end of the split compression ring.

According to another aspect of the present invention, a method of performing minimally invasive surgery includes: inserting an access port through the skin of a patient and positioning the access port adjacent or into a surgical target location, wherein the access port includes an elongate, tubular main body portion; a slot extending through the main body portion in a lengthwise direction, wherein the slot extends through a wall of the main body portion, from an outside surface of the main body portion to an inside surface of the main body portion; and a flap pivotally mounted to the main body portion at a distal end portion of the flap, the flap covering the slot in a closed configuration; inserting an instrument through the access port such that a distal end portion of the instrument extends into the surgical target location; rotating the flap away from the main body portion to an open configuration to expose the slot; and manipulating the instrument to pass a shaft portion of the instrument through at least a portion of the slot, wherein the instrument is manipulatable so as to angle the shaft relative to a longitudinal axis of the access port from a minimal angle of zero degrees to a maximum angle greater than that achievable without passing the shaft portion through at least a portion of the slot, and to any angle therebetween.

In at least one embodiment, the instrument is a second instrument, the method further including: inserting a first instrument through the access port while in a closed, configuration, prior to the rotating the flap, such that a distal end portion of the first instrument extends into the surgical target location; and performing work at the surgical target location with the distal end portion of the first instrument while the access port is in the closed configuration.

In at least one embodiment, the method further includes: removing the instrument from the access port; rotating the flap to the main body portion to the closed configuration; inserting a second instrument through the access port while in a closed, configuration, prior to the rotating the flap, such that a distal end portion of the first instrument extends into the surgical target location; and performing work at the surgical target location with the distal end portion of the second instrument while the access port is in the closed configuration.

In at least one embodiment, the method further includes: removing the instrument from the access port; rotating the flap to the main body portion to the closed configuration; and removing the access port from the patient while the access port is in the closed configuration.

In at least one embodiment, the method further includes fixing at least a portion of the access port to a stationary object.

In at least one embodiment, the surgical target location is in the spine of the patient.

In at least one embodiment, the surgical target location is an intervertebral disk space.

According to another aspect of the present invention, a method of performing minimally invasive surgery includes: inserting an access port in a closed configuration through the skin of a patient and positioning the access port adjacent or into a surgical target location, wherein the access port includes an elongate, tubular main body portion; a slot extending through the main body portion in a lengthwise direction, wherein the slot extends through a wall of the main body portion, from an outside surface of the main body portion to an inside surface of the main body portion; and a flap pivotally mounted to the main body portion at a distal end portion of the flap, the flap covering the slot in a closed configuration; rotating the flap away from the main body portion to an open configuration to expose the slot, wherein a proximal end of the flap extends out of the patient and is spaced away from the main body portion, and wherein a distal end portion inside the patient remains joined to the main body portion; performing a procedure through the access port with at least one instrument while the access port is in the open configuration; rotating the flap to the main body portion to resume the closed configuration; and removing the access port from the patient while in the closed configuration.

In at least one embodiment, the method further includes performing at least one task through the access port with an instrument while the access port is in the closed configuration.

These and other advantages and features of the invention will become apparent to those persons skilled in the art upon reading the details of the devices and methods as more fully described below.

### BRIEF DESCRIPTION OF THE DRAWINGS

FIG. 1A is a plan view of an access port according to an embodiment of the present invention.

FIG. 1B is a plan view of the access port of FIG. 1A after rotating it about its longitudinal axis by ninety degrees.

FIG. 1C illustrates a view from the proximal end of the access port of FIG. 1A.

FIG. 2A is a plan view of an access port that is a variant of the embodiment shown in FIG. 1A.

FIG. 2B is a plan view of the access port of FIG. 2A after rotating it about its longitudinal axis by ninety degrees.

FIG. 2C illustrates a view from the proximal end of the access port of FIG. 2A.

FIG. 3A is a view of an access port with a flap in an open configuration, according to an embodiment of the present invention.

FIG. 3B is a top view of the access port of FIG. 3A.

FIG. 3C is a bottom view of the access port of FIG. 3A.

FIG. 3D illustrates the angulation endpoints of an instrument inserted through an access port in a closed configuration, according to an embodiment of the present invention.

FIG. 3E illustrates the angulation endpoints of an instrument inserted through an access port in an open configuration, according to an embodiment of the present invention.

FIG. 4A shows a variant of the access port of FIG. 1A in which the distal end portion of the main body and distal end portion 1 of the flap are tapered.

FIG. 4B shows the access port of FIG. 4A in an open configuration.

FIG. 4C shows a variant of the access port of FIG. 1A in which the distal end portion of the main body and distal end portion of the flap include two tapered sections.

FIG. 5 illustrates a variant in which the flap and slot extend over only about 50% of the length of main body of an access port, according to an embodiment of the present invention.

FIG. 6 shows a variant of FIG. 3A in which skirts interconnect the sides of flap with the edges of the walls of the main body that define the slot.

FIG. 7 illustrates an access port according to another embodiment of the present invention in which a ratchet rack interconnects the flap with the edges of the walls of the main body that define the slot.

FIG. 8 illustrates a connector fixed to the main body of an access port according to an embodiment of the present invention.

FIG. 9 illustrates a variant of FIG. 8.

FIG. 10 is a partial view of an access port that is provided with a ring that is either fixed to, or can rotate within a limited range relative to the main body and to which a connector is fixed, according to an embodiment of the present invention.

FIG. 11A is a plan view of an access port according to another embodiment of the present invention.

FIG. 11B is a longitudinal section view of FIG. 11A taken along line 11B-11B, after insertion of light cables through light ports.

FIG. 12 is a partial view of an access port according to another embodiment of the present invention.

FIG. 13A is a cross sectional view of an access port according to an embodiment of the present invention.

FIG. 13B illustrates an illumination strip that can be used in the access port of FIG. 13A, according to an embodiment of the present invention.

FIG. 13C is a cross-sectional view of FIG. 13B taken along line 13C-13C.

FIGS. 14A-14B illustrate an access port with light guide assembly according to an embodiment of the present invention.

FIGS. 14C-14D illustrate steps in manufacturing a light guide assembly for use in an embodiment of the present invention.

FIGS. 15A-15K illustrate events that may be carried out using an access port according to an embodiment of the present invention.

### DETAILED DESCRIPTION OF THE INVENTION

Before the present devices and methods are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “an instrument” includes a plurality of such instruments and reference to “the implant” includes reference to one or more implants and equivalents thereof known to those skilled in the art, and so forth.

The publications discussed herein are provided solely for their disclosure prior to the filing date of the present application. The dates of publication provided may be different from the actual publication dates which may need to be independently confirmed.

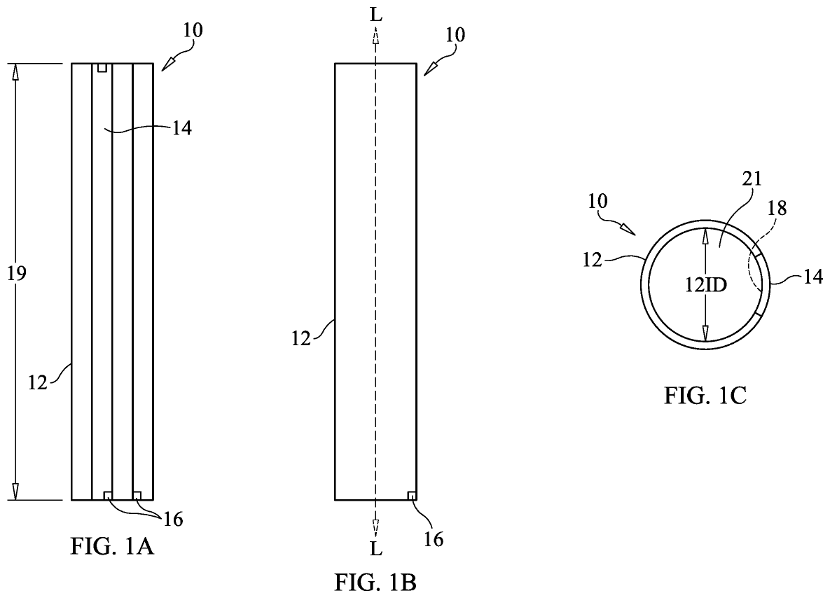

FIG. 1A is a plan view of an access port 10 according to an embodiment of the present invention. This and all other embodiments of access port described herein are configured and dimensioned for minimally invasive surgery, preferably, although not limited to, minimally invasive spinal surgery. Examples of preferred uses for ports 10 include posterior minimally invasive surgery to remove all or part of an intervertebral disk and perform a spinal fusion, which may include implantation of one or more implants and/or graft material into the intervertebral disk space; or lateral minimally invasive surgery to remove all or part of an intervertebral disk and perform a spinal fusion, which may include implantation of one or more implants and/or graft material into the intervertebral disk space.

Access port 10 includes a rigid, elongate main body portion 12 and a flap 14. Flap 14 is also preferably rigid, but may, alternatively, be flexible. In either case, flap 14, among other functions, acts as a mechanical barrier, and provides a heat barrier to insulate adjacent tissues from the heat generated by any heat-producing instrument (drills, diathermy, etc.)

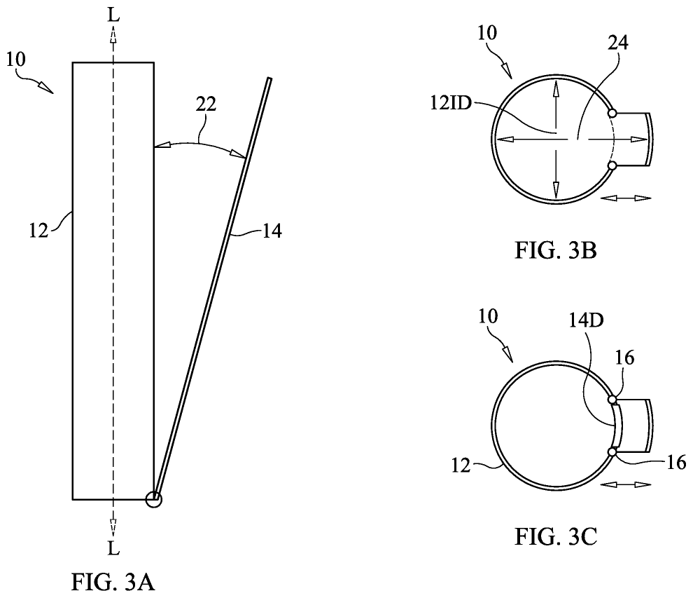

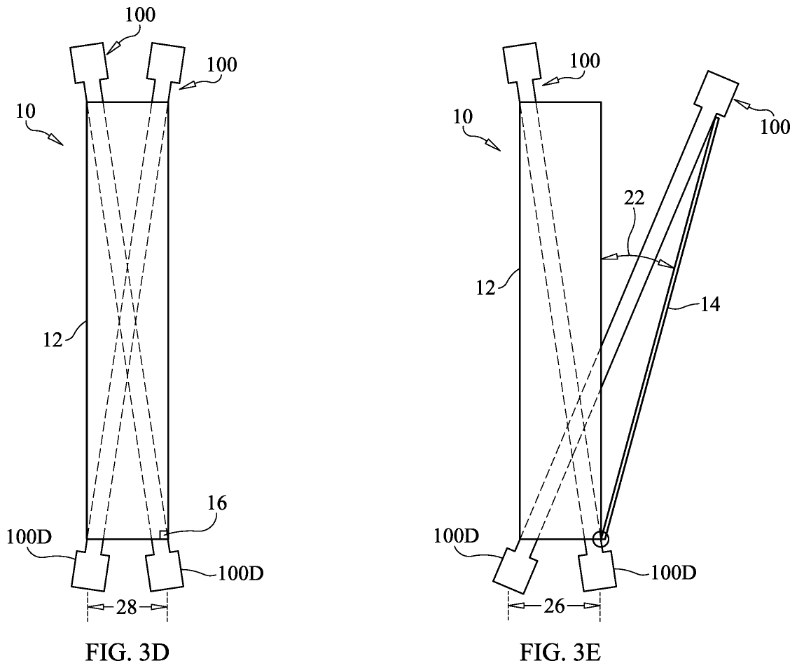

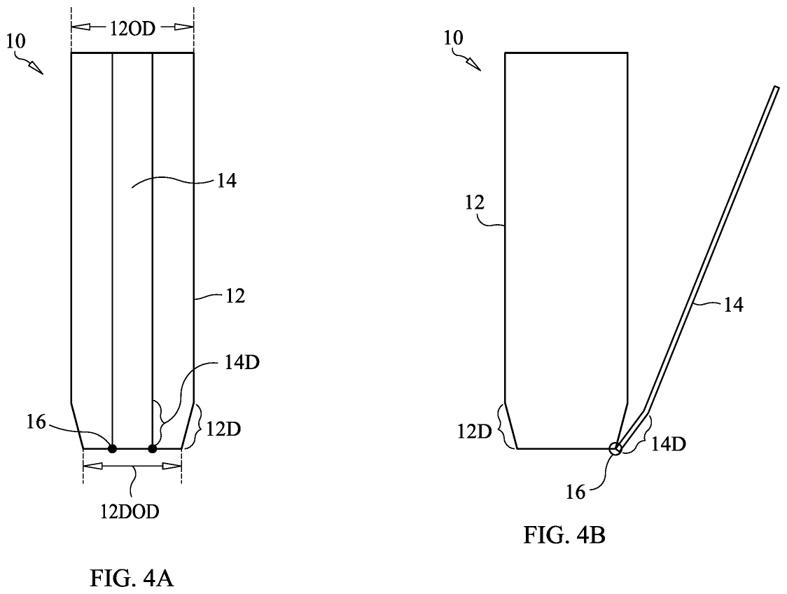

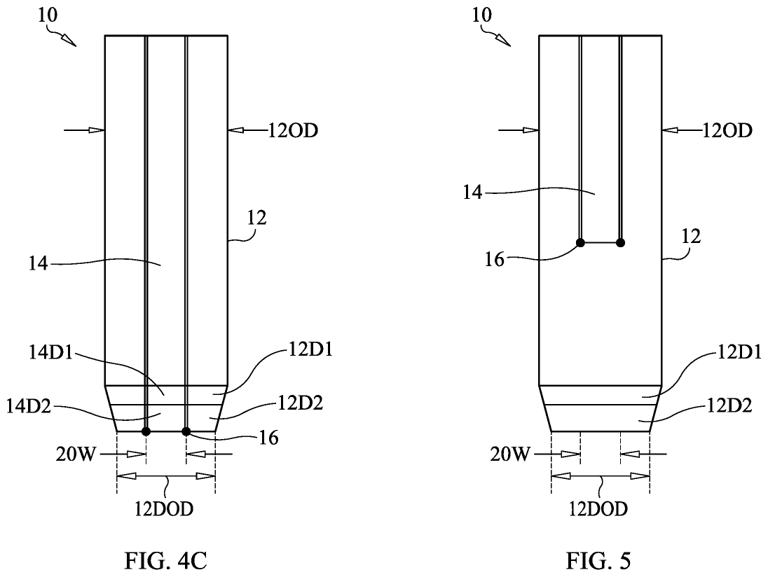

Main body portion 12 and flap 14 may be made of the same or different materials. As noted, main body portion 12 is made of rigid material so it has sufficient rigidity to maintain the its structural form during use. Body portion 12 and flap 14 may be made, for example, by machining from a monoblock of material, injection molding or three-dimensional (3D) printing. The main body portion 12 and flap may be made from the same material, or from different materials such as aluminum, stainless steel, titanium, aluminum alloys, titanium alloys or rigid polymer. Flexible polymer and/or metal may be used for making a flexible flap 14. The length 19 of port 10 typically is a value in the range of from about 30 mm to about 150 mm. The distal end portion 12D of port 10 may be continuous with the cross-dimensional conformation (e.g., cylindrical, oval or other shape tubular) of the main body 12, or may be tapered along one or more taper angles (as described in further detail in regard to the variants shown in FIGS. 4A-4C) to reduce the outside diameter of the port and facilitate insertion of the port through the tissues and into the tissue that it is used in, such as an intervertebral disk of the spine. Although the tapered feature is described as a variant of the embodiment shown in FIG. 1A, it is noted that this feature can be provided to any of the embodiments described herein. Further, the distal end portion 12D may include two or more tapered portions, as described below. The inside diameter 12ID (see FIG. 3B) of the untapered main body tube 12 may be of any size within a range of from about 8 mm to about 32 mm and may be tapered distally along one or more taper angles.

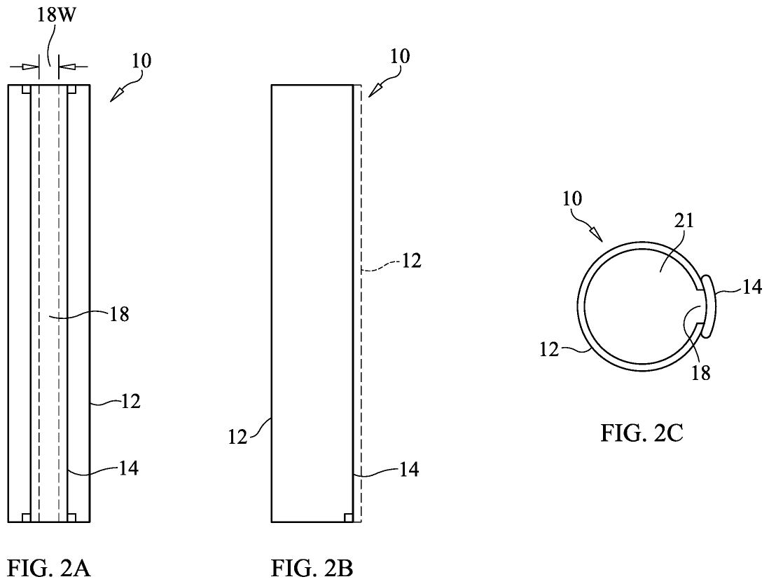

Flap 14 is attached to the main body portion 12 at their distal ends by connectors 16 such as hinges that allow the flap 14 to be pivoted away from the main body 12 to expose a slot 18 (see FIGS. 2A and 3A) to thereby extend a range of angulation of an instrument inserted into the access port 10. Hinges may reside either outside the main body portion 12 or distal of the distal edge of the main body portion 12.port. In the embodiment of FIGS. 1A-1B, flap 14, in the closed configuration as shown, fits in the slot 18 so as to close off the tubular main body 12 such that it has no exposed slot along the entire length thereof, and this functions as what is referred to as a closed tube. In all of the embodiments described herein, the access port in the closed configuration (closed tube) defines an annulus which is unobstructed by any other feature or component, such that the annulus (circular, oval, or whatever the cross-sectional shape of the access port 10 annulus) is defined in that shape all along the length of the main body 10. However, the access port 10 may include one or more ports in the main body 12 that allow insertion of instrumentation such as fiber optic lighting, or the like. In instances where the distal end is tapered, the annulus may be reduced in diameter (or other cross-sectional area) along the length of the tapered portion, but the same shape of shape of the annulus is maintained from the untapered portion. FIG. 1C illustrates a view from the proximal end of the access port of FIG. 1A, and shows that the circular (in this embodiment) cross-sectional shape of the annulus 21 is maintained throughout the length of the access port 10. The flap 14 can be configured to establish a positive securement, such as by a friction fit, ball and detent, or other mechanical securement mechanism that is releasable when the user manually applies relative opposing forces between the main body 12 and the flap 14. Thus, the user can simply pull on the flap 14 to release it from it secured position, so as to angulate it away from the main body 12. A “closed tube”, as used herein, refers to a tubular member that is open only at the proximal and distal ends of the annulus defined by the tube. Thus the walls of the tube are closed all along the lengths thereof and do not include any openings.

The access port 10 can be installed into tissue of a subject while in the closed configuration. This configuration not only reduces the profile of the port 10 to facilitate the insertion process, but it also prevents tissues from entering into the annulus of the port 10 during the insertion process, thereby ensuring a clear open annulus through which instruments can be inserted to perform a procedure. Thus the closed configuration provide more secure and complete protection of the tissues and neural structures from sharp, oscillating or rotating instruments during steps of a procedure using such instruments. As noted, the access port 10 can be used in the closed configuration to perform procedures where the flap 14, in the closed configuration, protects against damage to tissues surrounding the access port 10 during the performance of such procedures. For example, when using a sharp instrument, burr, or annulating knife, the closed flap 14 protects against cutting or abrasion of the tissues proximal to the surgical target site, while still allowing the instrument to cut or abrade tissues at the surgical target site which is distal to the distal opening of the access port 10. Additionally, the closed flap 14 can help prevent heat transfer to the surrounding features during such procedures.

FIG. 1B is a plan view of the access port 10 of FIG. 1A after rotating it about its longitudinal axis L-L by ninety degrees. Because the flap 14 fits within the slot 18 of the main body 12 in this embodiment, it can be flush with the walls of the main body 12 such that the access port 10 appears as a closed tube, as shown in FIG. 1B. FIG. 1C is a top view of the access port 10 of FIG. 1B. Flap 14 is shown fitting in and closing the gap 18 that exists when the flap 14 is in an open configuration. In the closed configuration, flap 14 is flush with the walls of the main body 12 and closes the gap 18. FIG. 1D is a perspective view of the access port 10 according to the embodiment of FIGS. 1A-1C.

FIG. 2A shows a variant of the access port 10 of FIG. 1A. The flap 14 of the access port 10 of FIG. 2A overlaps portions of the main body 12 wall while completely covering the slot 18 when in the closed configuration. As in the embodiment of FIG. 1A, the flap 14 forms a closed tube with the main body 12 when in the closed configuration, and otherwise functions in the same manner as described above with regard to the embodiment of FIG. 1A.

FIG. 2B is a plan view of the access port 10 of FIG. 2A after rotating it about its longitudinal axis by ninety degrees. Because the flap 14 overlaps the walls of the main body 12 in this embodiment, the overlapped portion of main body 12 is shown in phantom lines.

FIG. 2C illustrates a view from the proximal end of the access port of FIG. 2B, and shows that the circular (in this embodiment) cross-sectional shape of the annulus 21 is maintained throughout the length of the access port 10. Flap 14 is shown overlapping the walls of the main body 12 and closing the gap 18 that exists when the flap 14 is in an open configuration.

Preferably slot 18 extends nearly the full length of the main body 12 of the access port 10, but is closed off at the distal end of the main body 12 by the connection between the flap 14 and the main body. This closure of the distal end of the main body 12 by flap 14 so as to form a closed circumference (e.g., see FIG. 3C) at the distal end, prevents or reduces tissue creep into the access port 10, both during insertion of the access port 10, as well as during use of the access port 10. Alternatively, slot 18 may extend up to 99%, up to 90%, up to 80%, up to 70%, up to 60%, up to 50% or up to 40% of the full length of the main body 12, starting from the proximal end of the main body 12 and joined at a distal end of the slot 18 with a distal end of flap 14 in the same manner described above. In each case, flap 14 has a length at least as great as the slot 18, but may be longer, in which case flap 14 would extend proximally past the proximal end of the main body 12. In the embodiments of FIGS. 1A-3C, slot 18 extends through the full length of main body 12, except at the distal end of main body 12, where it is terminated (closed off) by the connection of the flap 14 to the main body 12. In these embodiments, slot 18 has a width 18W that is substantially invariant over the length of slot 18 to minimize reduction in rigidity of the main body 12 due to the formation of the slot 18, while affording a large range of angulation to tools used in conjunction with the access port 10. Width 18W may have a value in the range from 5 to 15 mm, or 6 to 12 mm. In at least one embodiment, width 18W was 8 mm Additionally main body 12 can be rotated about its longitudinal axis, either before or after insertion into the body so as to locate slot 18 at any angular position about the circumference of the main body desired. This greatly increases the area that can be reached by the working end of a tool inserted through the access port 10. The width 18W may be a value in the range of from about 8 mm to about 18 mm. The outside diameter 120D of the non-tapered portion of main body 12 may be a value in the range of from about 8.5 mm to about 34.5 mm. In tapered embodiment, the outside diameter 12DOD of the distal end of end portion 12D may be a value in the range of from about 6 mm to about 32 mm.

FIG. 3A shows the embodiment of FIG. 1B in an open configuration. It is noted that the embodiment of FIG. 2B also appears as shown in FIG. 3A when in an open configuration. By holding the main body 12 stationary (either by hand or by fixing it to a relatively stationary object), and applying a traction force to the flap 14, such as by grasping the proximal end portion of the flap 14 either by hand or with an instrument and pulling it away from the main body 12, the flap 14 can be rotated away from the main body 12 as shown. This rotation is achieved by rotation about the pivot point defined by hinges 16 or other mechanical expedient that attaches the distal end of the flap 14 to the main body 12 and allows rotation of one relative to the other. The access port can be configured to allow angulation of the flap 14 relative to the longitudinal axis L-L of the main body 12 by any angle 22 within a range from 0 to 65 degrees, typically from 0 to 45 degrees, or within a range from 0 to 30 degrees.

The flap 14 can be connected to the main body 12 and configured so that it can be selectively opened to any angle within the range of angulation that is possible. For example, hinge 16 may be provided as a torque and positioning hinge, which functions like the hinge of a laptop computer that allows the screen to be stably placed at a desired angulation relative to the keyboard. This allows the flap 14 to assume a stable position at any angle between 0 degree and the maximum angulation allowed, until a user again applies a force to close the flap 14 or reposition it to a different angle.

In an open configuration where angle 22 is greater than 0 degrees, a greater range of angulation of instruments used in an access port 10 is provided, compared to the range of angulation allowed by the access port 10 in the closed configuration. This results in a relative greater length along the target area (distal of the distal end of the access port 10) that can be affected by a working end of an instrument inserted through the access port 10 as it is moved along the greater range of angulation that is provided. Additionally the access port 10 can be rotated about is longitudinal axis, so that that the greater length of working distance that is available can result in a greater area (such as a circle, when the access port is cylindrical) of the target that can be reached by the working end of the instrument, as the access port can be rotated to any orientation 360 degrees about its longitudinal axis. FIG. 3B is a proximal end view of FIG. 3A that shows the greater distance 24 provided by the flap 14 in the open configuration that allows greater angulation of an instrument used in the access port 10, compared to the distance 12ID that is available when the access port 10 is in the closed configuration. FIG. 3C is a distal end view of FIG. 3A that shows that the distal end of the access port 10 remains closed, as the distal end 14D remains joined to the distal end of the main body 12 so that the distal end of the access port 10 is continuously confined around its entire perimeter. FIG. 3E illustrates the greater distance 26 that a working end 100D of an instrument 100 inserted through the access port 10 can travel when the flap 14 is in an open configuration, compared to the distance 28 that the working end 100D of the instrument 100 inserted through the access port 10 can travel when the flap 14 is in the closed configuration shown in FIG. 3D, because of the increased range of angulation afforded to the instrument 100 when used in the access port 10 in the open configuration.

FIG. 4A shows a variant of the access port of FIG. 1A in which the distal end portion 12D of the main body 12 and distal end portion 14D of the flap are tapered, so that the distal end portion of the access port 10 is tapered when in the closed configuration.

FIG. 4B illustrates the access port 10 of FIG. 4A having been rotated by 90 degrees about its longitudinal axis, and with the flap 14 having been pivoted away from the main body 12 to an open configuration of the device. FIG. 4B shows the tapered section 14D of the flap that conforms to the tapering of the tapered section 12D when in the closed configuration.

The distal end portion 12D of port 10 may be tapered along one or more taper angles to reduce the outside diameter of the distal end portion of the port and facilitate insertion of the port through the tissues along the path to the target tissue and into the target tissue, such as an intervertebral disk or other target tissue. FIG. 4A shows that the outside diameter 120D of the main body 12 is greater than the outside diameter 12DOD at the distal end of the main body 12 that results from the tapering.

FIG. 4C shows a variant of the access port of FIG. 1A in which the distal end portion 12D of the main body 12 and distal end portion 14D of the flap include two tapered sections, with first tapered sections 12D1, 14D1 having a taper angle less than the taper angle of the second tapered sections 12D2, 14D2. The inside diameter 12ID (see FIG. 1C) of the main body tube 12 may be of any size within a range of from about 8 mm to about 32 mm and is typically tapered distally along one or more taper angles. The inside diameter of the tapered distal end portion may have a value at the distal end of the main body portion in the range from about 10 mm to about 24 mm. In one embodiment the inside diameter at the tapered distal end of main body 12 was 18 mm. The width 20W of the flap 14 may have a value in the range from 5 to 15 mm, or 6 to 12 mm. In at least one embodiment, width 20W was about 8 mm. For embodiments in which the flap overlaps portions of the wall of the main body 12 around the slot 18, width 20W may have a value greater than those embodiment in which the flap 14 fits into the slot 18 to close it. For example, the width 20W of the overlapping embodiments may be in a range from 6 mm to 17 mm. In at least one embodiment, the width 20W was 10 mm and width 18W was 18 mm.

It is noted that the embodiment of FIGS. 2A-2B and all other non-tapered embodiments described herein can alternatively be tapered in the same manner as described with regard to FIGS. 4A-4C.

FIG. 5 illustrates a variant in which flap 14 and slot 18 extend over only about 50% of the length of main body 12. Thus the distal end of flap 14 is attached to the main body at 16, about halfway along the length of the main body 12 and flap 14 extends up to the proximal end of the main body 12. It is noted that other variants can have a flap 14 and slot 18 of other lengths, as noted below. It is further noted that these variants can be applied to the overlapping flap 14 embodiment of FIGS. 2A-2B and any other embodiment (tapered or non-tapered) described herein that has a slot 18 and flap 14 that extend nearly the entire length of the main body 12.

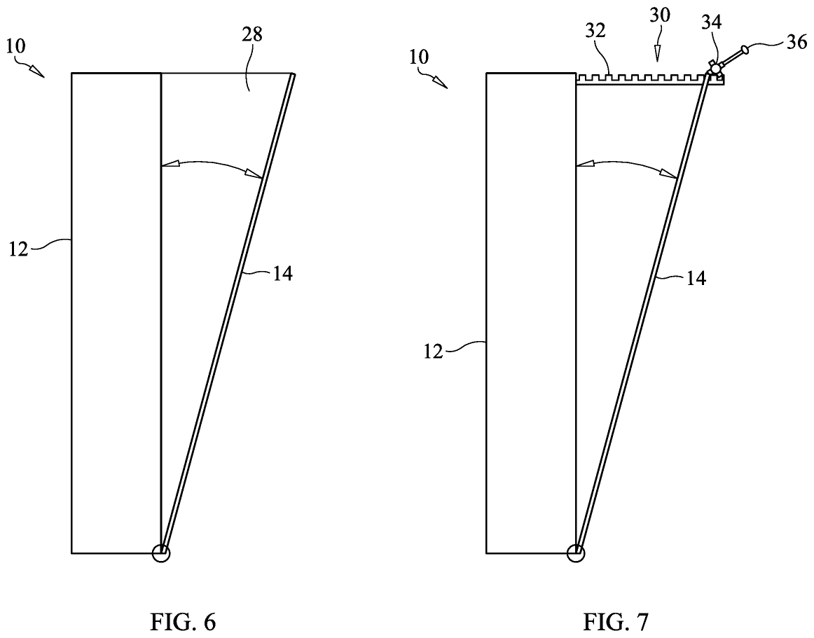

FIG. 6 shows a variant of FIG. 3A in which access port 10 skirts 28 interconnect the sides of flap 14 with the edges of the walls of main body 12 that define the slot 18. Skirts 28 are flexible so that they do not interfere with the angulation of the flap 14 relative to the main body 12. Skirt 28 close off the triangular openings that present between the flap and the main body walls when the flap 14 is placed in an open configuration. FIG. 6 shows skirt 28 on one side of the flap 14 and another skirt 28 can be configured on the opposite side of the flap 14. Skirts 28 therefore prevent entrance of tissues, fluids, etc. into the access port through spaces between the main body 12 and the open flap 14. Skirts 28 may be made from a flexible polymer, such as an ePTFE material, non-molded fluorinated ethylene propylene (FEY), a polyester knitted fabric, a polyester velour, a polypropylene felt, a woven or braided fabric, a non-woven fabric, porous material, other textile material, or other flexible, biocompatible material that can function for the state purpose. Skirts 28 can be provided with any of the access ports described herein, tapered or non-tapered.

FIG. 7 illustrates an access port 10 according to an embodiment of the present invention. In this embodiment, an angulation control mechanism 30 is provided that can be actuated to drive the flap 14 in a controlled manner such that the flap can be opened or closed via the mechanism 30 to position and maintain the angulation of the flap 14 relative to the longitudinal axis L-L of the main body 12 at any angle 22 within the range of possible angles that the flap 14 can assume relative to the main body 12. In one non-limiting example, the angulation control mechanism may be a ratchet mechanism (ratchet rack) having a rack 32 and pinion 34 controlled by a handle 36 to ratchet the pinion 34 along the rack 32. The rack 32 extends radially outwardly from the main body by a sufficient distance to allow angulation of the flap 14 up to the maximum angle (such as 45 degrees or 30 degrees, or some other predefined maximum angle) 22, with the pinion being mounted to the proximal end portion of the flap 14. Alternative angulation control mechanisms 30 may include a screw drive, a mechanism with a ball and a series of detents, or the like. Although shown in FIG. 7 in used with a non-tapered access port 10 of the type shown in FIG. 1A, it is noted that angulation control mechanism 30 can be provided with any of the embodiments described herein. Also, the use of an angulation control mechanism 30 does not preclude the use of a torque and positioning hinge as described herein, although the torque and positioning hinge would typically not be required when employing an angulation control mechanism.

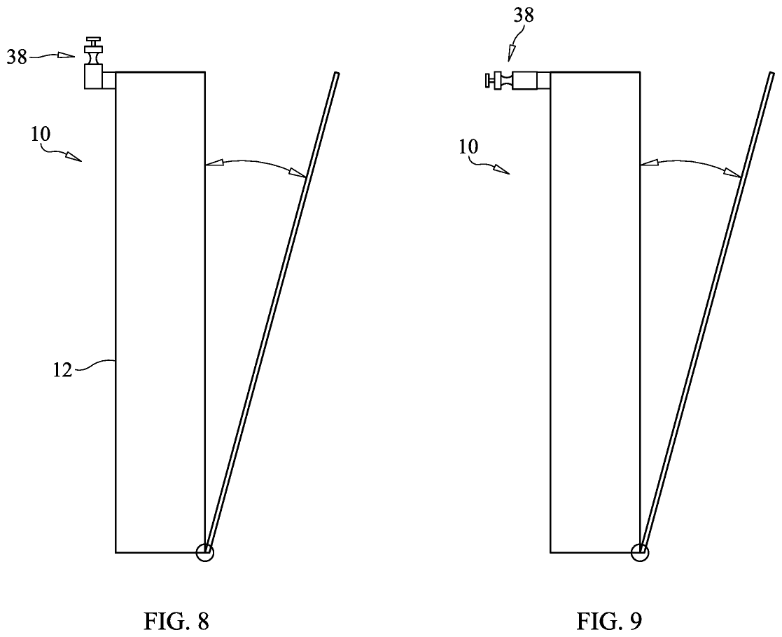

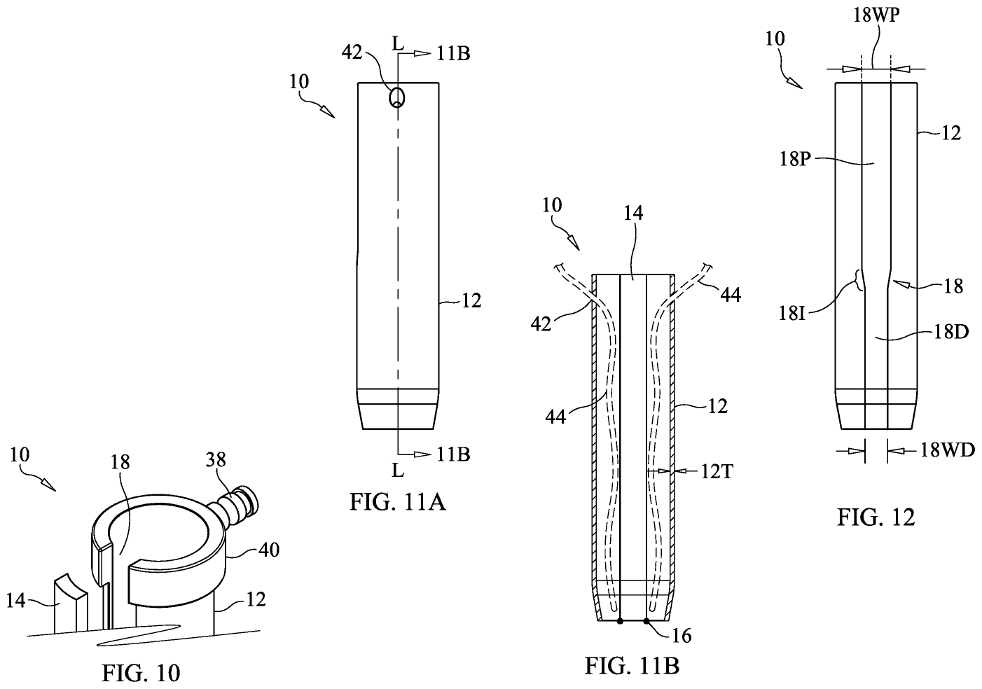

FIG. 8 illustrates a connector 38 fixed to the main body 12 of access port 10 according to an embodiment of the present invention. Connector 38 is connected to the proximal end or proximal end portion of the main body 12 and extends proximally from main body 12 in the embodiment of FIG. 8. Alternatively, connector 38 may be connected to the proximal end or proximal end portion of the main body 12 and extend radially from the main body 12, as shown in FIG. 9. Further alternatively, the connector 38 may extend from the proximal end or proximal end portion of main body 12 in a direction at any angle relative to the longitudinal axis L-L of the main body 12. Still further alternatively, as shown in the partial view of FIG. 10, access port 10 can be provided with a ring 40 that is either fixed to, or can rotate within a limited range relative to main body 12 and to which connector 38 is fixed. These types of arrangements are described in U.S. Patent Application Publication No. 2016/0270816, which is hereby incorporated herein, in its entirety, by reference thereto. Connector 38 is configured to connect and fix access port 10 to a stationary object, such as an operating table or other stationary object.

FIG. 11A is a plan view of an access port 10 according to another embodiment of the present invention. The newly described features can likewise be applied to all other embodiments described herein. In the embodiment of FIGS. 11A-11B a pair of light ports 42 are provided through the wall of the main body 12 at a proximal end portion of the main body 12 to allow light cables 44 (such as fiber optic lighting, LED lighting, or the like) to be inserted therethrough and extend anywhere along the inner length of access port 10, up to, or even into the surgical site, to illuminate the workspace. However, typically the light cables are inserted through the light ports just enough so that the distal tips of the light cables 44 stay substantially flush with the inside openings of the light ports 42 and inner wall of the main body 12 to illuminate the inside of the main body 12.

It is noted that the present invention is not limited to two light ports 42, as one light port 42 or more than two light ports 42 could be provided. Light ports 42 angle down toward the distal end of the access port 10 as illustrated in the longitudinal sectional view of FIG. 11B. In the embodiment of FIGS. 11A-11B, the light ports 42 are conical, with a larger inside diameter at the outer surface of the main body 12 than the inside diameter of the port 42 at the inner surface of the main body 12. For example, the inside diameter at the outer surface may be about 5 mm when the inside diameter at the inner surface is about 4 mm. These diameters may vary from embodiment to embodiment, and are typically in a range of about 2 mm to about 7 mm. Alternatively, ports 42 could be made cylindrical or have a non-circular cross-sectional shape. The thickness 12T of the wall of the main body 12 in FIGS. 11A-11B is about 1.5 mm, but may be any value within a range of from about 0.5 mm to about 2.5 mm. These thickness values can also be applied to all other embodiments of the main bodies 12 of the access ports 10 described herein.

FIG. 12 is a partial view of an access port 10 according to another embodiment of the present invention. The access port 10 of FIG. 12 is shown without the flap 14 for optimizing the viewing of the slot 18. As shown in FIG. 12, the slot 18 tapers from a proximal end portion to a distal end portion of the access device 10, such that the proximal end portion 18P is wider than the distal end portion 18D. This provides more strength in the main body 12 at the distal end portion relative to the proximal end portion, to better resist yielding under compression forces experienced during use. It is noted that all other embodiments described herein that use a non-tapering slot 18 could alternatively be provided with a tapering slot 18 as described here. In FIG. 12, the width 18WP of the proximal end portion of slot 18 is about 14 mm and is substantially constant over the length of the proximal end portion of slot 18, but could be any value in a range of from about 10 mm to about 18 mm, and the width 18WD of the distal end portion of slot 18 is about 12 mm and is substantially constant over the length of the distal end portion of slot 20, but could be any value in a range of from about 8 mm to about 16 mm. An intermediate portion 18I of the slot 18 varies in width so as to join the proximal and distal end portions of the slot 18. The length of the proximal end portion of the slot 18 is typically greater than the length of the intermediate portion 811, but need not be. The length of the distal end portion 18D of the slot 18 is also typically greater than the length of the intermediate portion 18I, but need not be. Typically the intermediate portion is provided as a transition and is less than ¼ the length of either the proximal end portion or the distal end portion of the slot 20. However, proximal end portion 18P may have a length in the range of ten percent to ninety nine percent of the length of main body 12, distal end portion may have a length in the range of ten percent to ninety nine percent of the length of main body 12 and intermediate portion may have a length in the range of one percent to eighty percent of the length of main body 12.

In embodiments where the flap 14 closes flush with the walls of the main body 12 of the access port, the flap 14 will have proximal end portion, intermediate portion and distal end portions that have length dimensions the same as those of 18P, 18I and 18D, respectively and which have width dimensions that are about the same, or slightly less than those of 18P, 18I and 18D, respectively, so that the flap 14 can be securely received in the slot 18 to close the access port 10. In embodiments where the flap overlaps the walls of the main body 12 that define the slot 18, the flap will have a length dimension that is the same or about the same as the length of the main body 12, but the flap need not have tapered width dimensions, as long as the width is sufficient to overlap the slot at the widest (proximal end) portion. Alternatively, flap 14 can be tapered to have a proximal portion that has a length equal to or about the same as the length of 18P and a width slightly greater than the width of 18P to allow establishment of the overlap, an intermediate portion that has a length equal to or about the same as the length of 18I and a width slightly greater than the width of 18IP to allow establishment of the overlap, and a distal portion that has a length equal to or about the same as the length of 18D and a width slightly greater than the width of 18D to allow establishment of the overlap. Further optionally, the flap can have any shape as long as it can function to overlap the slot 18 along its entire length.

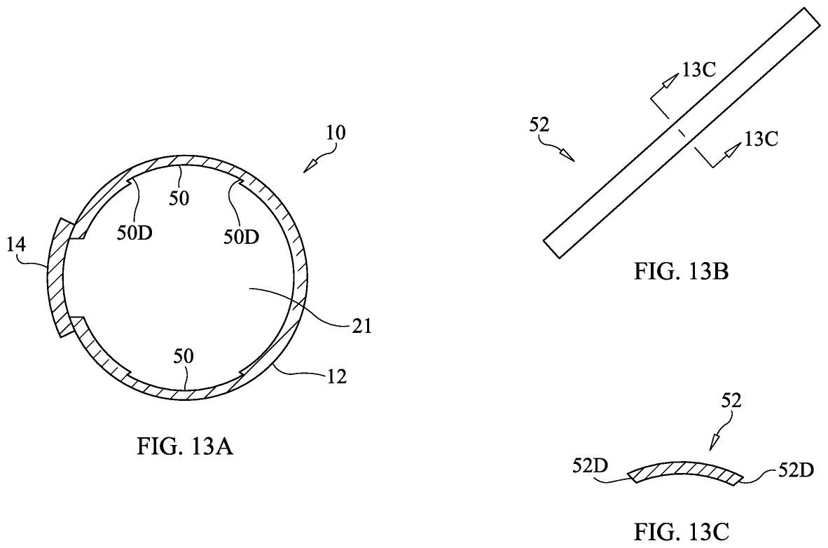

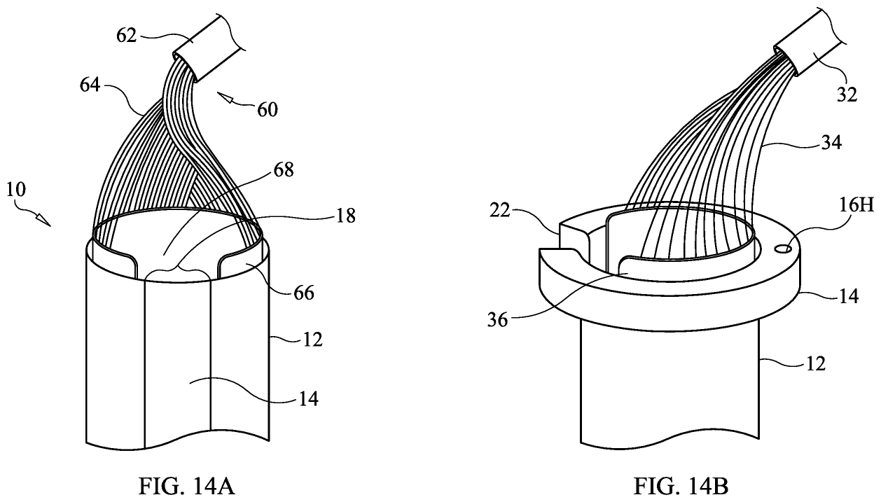

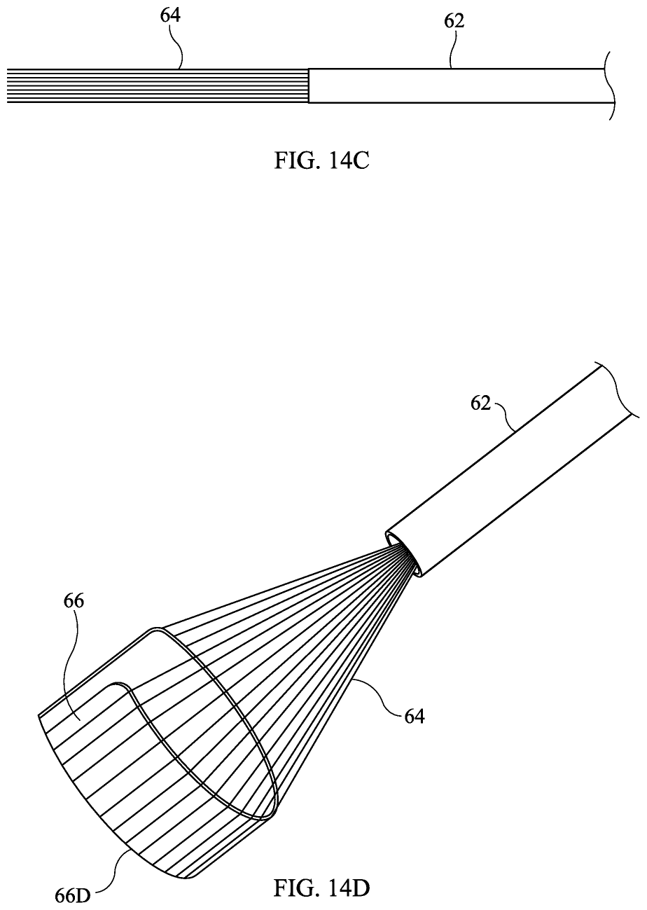

FIG. 13A is a cross sectional view of an access port 10 according to an embodiment of the present invention, in which the flap 14 is shown in the closed configuration, and in which cutouts 50 are provided in the inner wall of the main body 12. Cutouts 50 may extend the entire length of the main body 12, or only a portion thereof. Preferably, the cutouts 50 extend over substantially the full length of the main body 12. Illumination strips 52 (see FIGS. 13B-13C) can be provided as configured and shaped to slide into and mate with the cutouts 50, to provide illumination within the access port 10 and into the surgical target area during use of the access port 10. Preferably the cutouts 50 and illumination strips 52 are dovetailed (see 50D, FIGS. 13A and 52D, FIG. 13C, respectively) to more securely join the components together. Advantageously, the illumination strips 52 are sized and shaped to as to be flush with the inner wall of the main body 12, so as not to provide any obstruction to the annulus 21 of the access port 10. The cross-sectional view of illumination strip 52 in FIG. 13C shows the curved, mating dovetail shape of the illumination strip 52 in cross section. In the view of FIG. 13B, it can be seen that the illumination strip is typically an elongate, rectangular shape in the plan view. Illumination strips 52 may be commercially available illumination strips such as LightMat Ultra-Thin strips, Model No. UA2550, available from Lumitex in Maryland. It is further noted here, that all embodiments described herein could include the cutouts 50 and illumination strips 52 in the same manner as described with regard to the embodiment of FIGS. 13A-13C. It is further noted that only one cutout 50 and illumination strip 52 could be provided, or more than two cutouts 50 and more than two illumination strips 52 could be provided.

FIGS. 14A-14B illustrate an access port 10 according to another embodiment of the present invention. Although illustrate with regard to an embodiment in which flap 14 closes flush with the main body 12, it is noted that any of the embodiments and variants described herein can be provided with the new features described with regard to FIGS. 14A-14B. A light guide assembly 60 is shown installed in the opening of the main body 12. Light guide assembly 60 includes a fiber optic cable 62 optical fibers/fiber optic filaments 64 and compression ring 66. Compression ring 66 is slotted, with slot 68 having a width at least equal to and typically greater than (greater than, in the embodiment shown in FIGS. 14A-14B) the width of the slot 18 in main body 12, as best shown in FIG. 14A.

To manufacture the light guide assembly 60, the optical fibers 64 of a fiber optic cable 62 are exposed along a distal end portion of the fiber optic cable 62 as illustrated in FIG. 14C. The optical fibers 64 are next fanned out and attached around the compression ring 66 in an even distribution, as illustrated in FIG. 14D. The distal ends of the optical fibers 64 end at or just short of the distal end 66D of compression ring 66. The optical fibers 64 can be attached using adhesive, adhesive tape, or other equivalent that is sufficient to maintain adherence of the optical fibers 64 to the compression ring 66 even under sterilization environments. The optical fibers 64 of the fiber optic cable 60 are very numerous, typically in the range of about 900-1200 optical fibers, but could be more or less. This large number of optical fibers 64 when spread out form a very continuous, even light source around the inner circumference of the main body 12 as they project light along the inner walls (typically parallel to, but could be angled) of the tubular opening through the main body 12.

To install the light guide assembly 60 to the access port 10, the compression ring 66 is squeezed to temporarily reduce the outside diameter thereof to allow it to be slid into the proximal end of the opening of main body 12. Thereafter, the squeezing compression force is released, allowing the compression ring to resiliently return to its unbiased, larger outside diameter. This fixes the compression ring 66 against the inner wall of the main body 12, forming a compression fit that maintains the light guide assembly 60 in the desired position relative to the main body 12. The optical fibers are positioned so as to direct light evenly all along the inside opening of the main body 12, thereby providing even lighting all along the length of the opening and eliminating any shadows or “dead spots” that occur with other lighting arrangements. Alternatively, the compression ring 66 can be permanently fixed to the inner wall of the opening of the main body 12, such as by adhesives, welding or the like. Further alternatively, the optical fibers 64 could be adhered to the proximal end portion of the opening of the main body 12 without the compression ring 66. However, the compression fitting of the ring 66 to the inner wall of the main body 12 provides the advantages that it can be removed, so that the access port 10 can be used without the light guide assembly 60 if desired and/or can be removed to simplify sterilization procedures.

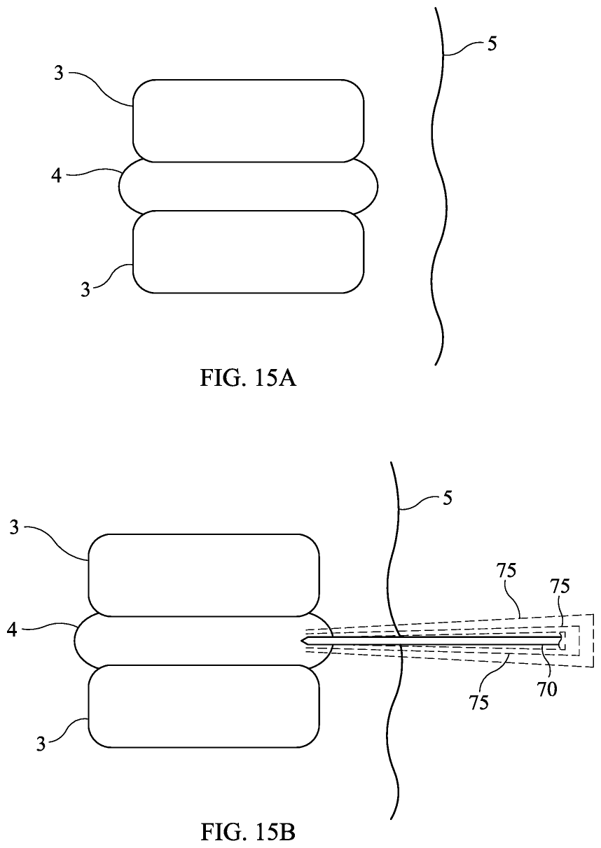

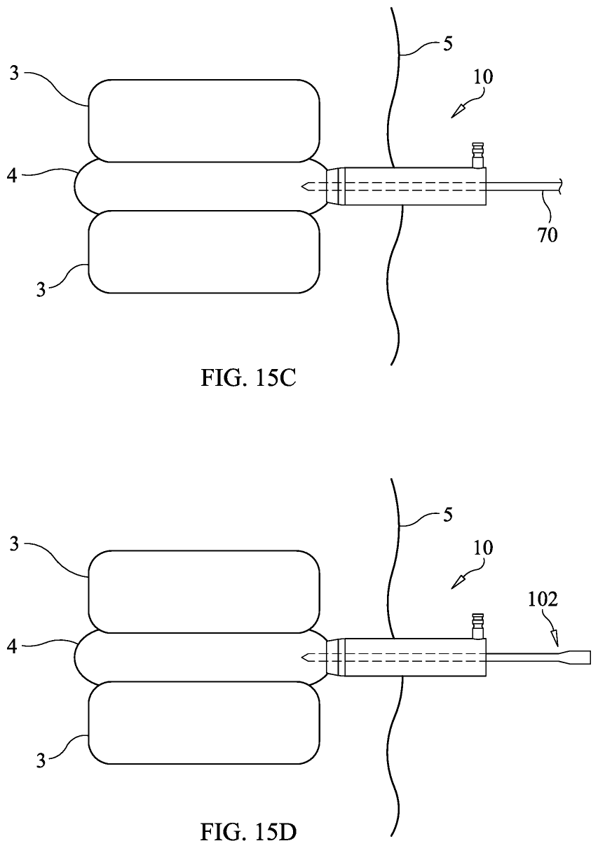

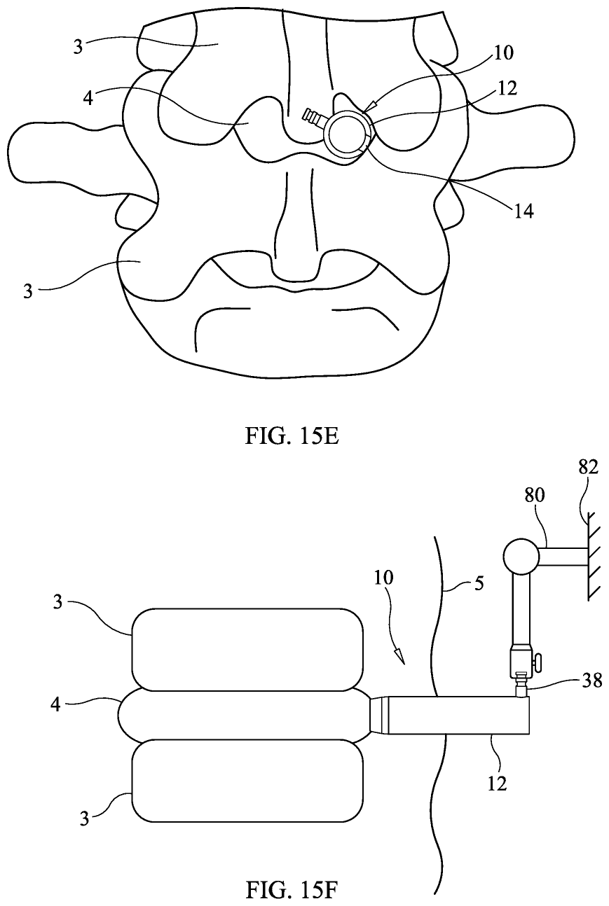

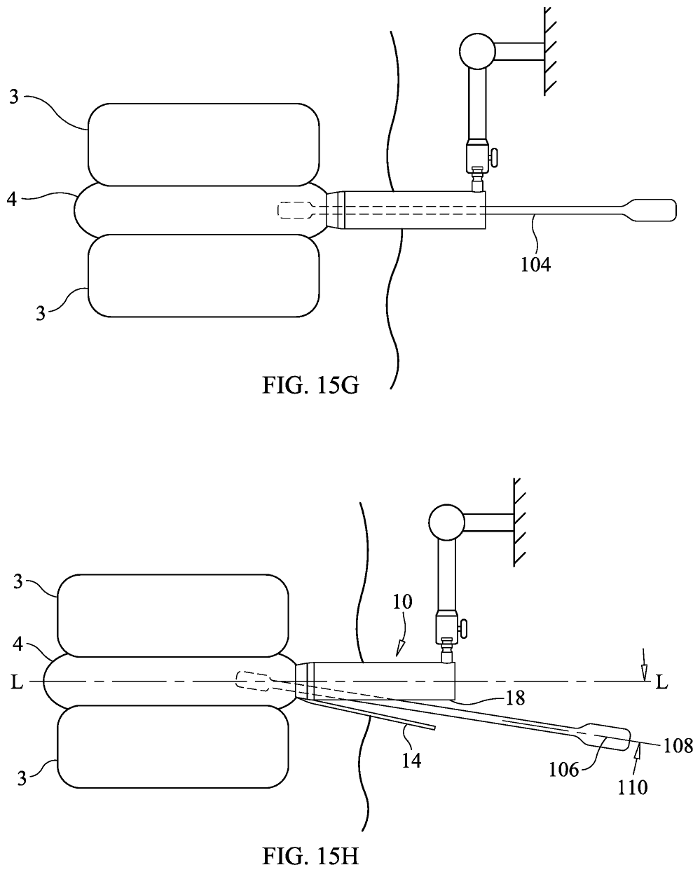

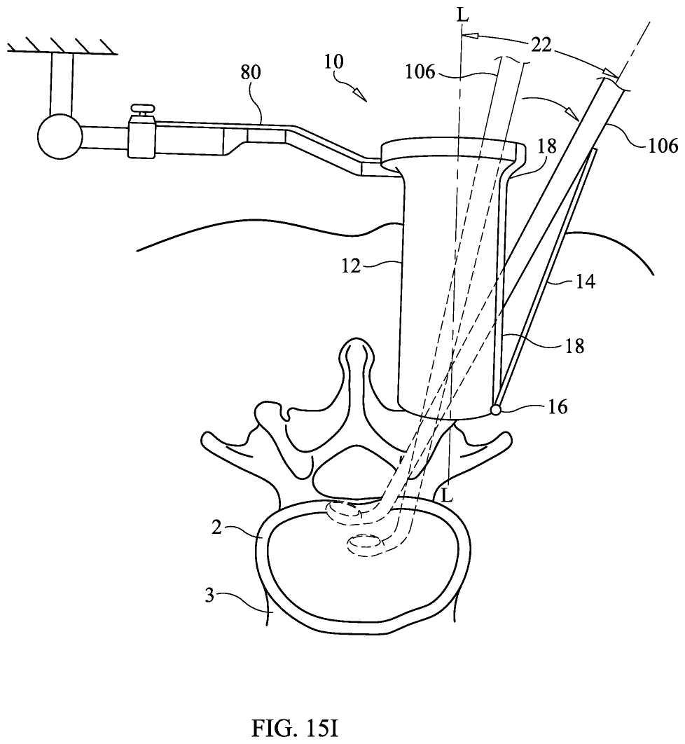

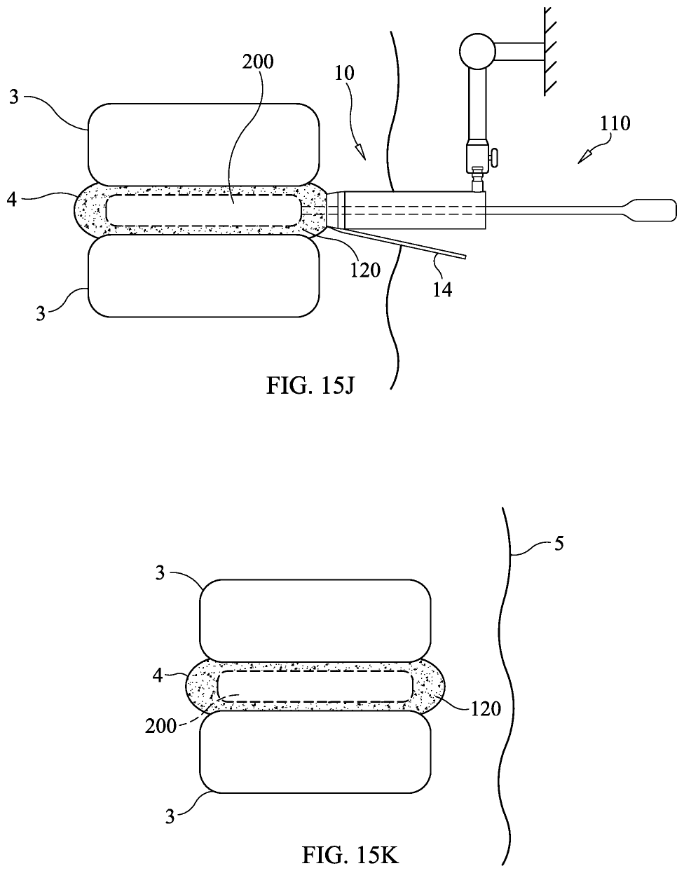

FIGS. 15A-15K illustrate events that may be carried out using an access port 10 according to an embodiment of the present invention. FIG. 15A schematically illustrates a side view of pair of adjacent vertebrae 3, an intervertebral disk 4 between the vertebrae 3 and skin 5 of the back of a patient in which a procedure is to be carried out. Typically, an incision having a length in the range of about 15 mm to about 18 mm is made through the skin 5 and the fascia and other tissues underlying the skin are manipulated with tools to provide a minimally invasive opening to the intervertebral disk 4 (surgical target location). Optionally, a K-wire or other guide 70 can be first inserted through the opening and into the intervertebral disk 4, as illustrated in FIG. 15B, and then dilators 75 of increasing diameter can be subsequently used to increase the size of the pathway leading to the surgical target location. Alternatively, the access port 10 can be installed without the use of a K wire or guide 70, for a posterior procedure, like what is illustrated in FIGS. 15A-15K. When the access port 10 is used for a lateral procedure (i.e., access port is inserted laterally of the disk 4, rather that posteriorly), a K-wire or other guide 70 is typically used to guide insertion of the access port 10. Dilators 75 can also be used in any of these approaches.

FIG. 15C illustrates the access port 10 having been inserted through the opening of the skin 5, with or without guidance by guide 70, and installed so that a distal end of the access port 10 enters or is in contact with the intervertebral disk 4. A series of dilators can be used around a firstly-inserted Jamshidi Needle to prepare for the introduction of the access port 10. Typically, but not necessarily, it is sufficient for the distal end of the access port 10 to contact the disk 4 externally, but not enter the disk 4 space. In the optional case where a guide 70 is used, the guide is next removed from the patient and from within the access port 10, as shown in FIG. 15D. FIG. 15D shows that one or more tools can be used to perform tasks while the access port 10 is in the closed configuration. For example, FIG. 15D illustrates an annulating knife 102 being used to cut into the disk 4. The access port 10 remains in the closed configuration to better protect against cutting any tissues located between the skin 5 and the disk 4 as the sharp features of the instrument 102 are passed through the annulus of the access port 10. Tasks of this type may be performed before or after fixing the access port 10 to a stationary object via connector 38. Further optionally, for an access port 10 that is not provided with a connector 38, the access port may be held stationary by hand.

FIG. 15E is a top view of FIG. 15E, after having removed instrument 102. In this view, the feet of the patient are in the direction of the bottom of the drawing sheet and the surgeon would be located such that flap 14 faces toward the direction of the surgeon. The connector 38, if present, is next connected to an interconnecting rod or linkage 80 (see FIG. 15F) that is also connected to a stationary object 82, such as the operating table or other stationary object, so that the access port 10 is fixed. The access port can later be rotated, if desired, by loosening one or more connections, reorienting the access port 10 to the rotational orientation desired, and then again fixing the connector to render the access device 10 stationary.

FIG. 15G illustrates an instrument 104 being inserted into the disk 4 space in a procedure to remove a herniated disk prior to implantation. Instruments 90 that may be used in this procedure include, but are not limited to: rongeurs, rasps, curettes, and cutting instruments. As noted, rasps, cutting instruments and the like that may injure tissue in between the surgical target area and a location outside the patient may be used while the access port 10 is in a closed configuration, as illustrate in FIGS. 15D and 15G. Suction may also be used to help remove finer particulates and liquids, such as blood, etc. In order to extend the range and angulation over which the working ends of instruments 106 can operate, for instruments 106 that are less likely to cause damage to intervening tissues along the path from outside the patient and up to the surgical target location, the surgeon can manipulate the access port 10 to an open configuration as illustrated in FIG. 15H. This can be done by any of the methods described previously, or, alternatively, the instrument 106 can be used to apply force to the flap 14 to open it. By placing the access port 10 in an open configuration, slot 18 is exposed so that the surgeon can work the proximal end portion of the shaft of the instrument 106 through the slot 18 of the access port 10 as illustrated in FIG. 15H. The open flap 14 still provides some degree of tissue protection of the tissues that it abuts. If skirts 28 are used, this further helps to prevent entrance of tissues and bodily fluids into the annulus of the access port 10. Slot 18 provides the ability to angle the instrument 106 relative to the access port 10, such that the longitudinal axis 108 of the instrument shaft forms an angle 110 with the longitudinal axis L-L of the access port 10 up to 65 degrees or more, typically within a range of about 25 degrees to about 60 degrees, and in other instances, a range of from about 15 degrees to 20 degrees is sufficient. Any angle up to the maximum angle allowed by the maximum angle that the flap 14 can open, can be achieved by the instrument 106. As noted previously, the access port 10 can be repositioned (when not connected to a stationary object) by rotating it about its longitudinal axis to further extend the range of the space that the working ends of the instruments 106 can reach.

FIG. 15I illustrates the large angulation 22 that the instrument 106 is capable of achieving relative to the longitudinal axis L-L of the access port because of the opening of the continuous slot 18 when flap 14 is angled away from the main body 12. The ability to rotate the access port 10 about its longitudinal axis L-L as described further increases the area that the working end of the instrument 106 can reach. Instruments can be used in this manner to remove disk material, cleaning of the disk space, implantation of bone graft material and/or implants, etc. Slot 18 can be oriented at any angle about the circumference of the main body 12 relative to the cranial-caudal axis, and further can be repositioned to any other angle even after insertion, fixation and working with tools 106. This increases the versatility and range of access provided by the access port 10.

After the disk 4 space has been sufficiently cleared and suctioned and all instrumentation 106 has been removed from the access port 10, one or more implants 200 (typically one or two, inserted sequentially) can be inserted through access port 10 and implanted in the intervertebral disk 4 space, using an inserter instrument 110, as illustrated in FIG. 15J. Additionally, graft material 120, such as bone particles or chips, demineralized bone matrix (DBM), paste, bone morphogenetic protein (BMP) substrates or any other bone graft expanders, or other substances designed to encourage bone ingrowth into the disk 4 space to facilitate the fusion may be implanted. Facilitation of the placement of graft material can be improved when the access port is in the open configuration as shown in FIG. 15J, while the placement of implants 200 may be performed with the access port 10 in the open or closed configuration. Further details about implants and graft material can be found in U.S. Pat. No. 8,956,414, issued Feb. 17, 2015 and U.S. Pat. No. 8,906,097, issued Dec. 9, 2014, both of which are hereby incorporated herein, in their entireties, by reference thereto.

After completion of the implantation of the implant(s) 200 and, optionally, bone graft material 120, the inserter 110 is detached from the last implant 200 implanted and removed from the access port 10 and then the access port 10 is removed and the patient is closed according to standard procedures. FIG. 15K schematically illustrates the surgical target location after completion of the closure.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

That which is claimed is:

1. An access port configured and dimensioned for use in minimally invasive surgery, said access port comprising:
an elongate, tubular main body portion having a first proximal end portion and a first distal end portion, said distal end portion being configured to be inserted into a patient and at least a portion of said proximal end portion being configured to remain outside of the patient during use;
a slot extending over a length of said main body portion in a lengthwise direction; and
a flap having a length at least as great as a length of the slot, said flap having a second proximal end portion and a second distal end portion;
wherein said flap is pivotally joined to said main body portion by said second distal end portion being pivotally joined to said first distal end portion;
wherein said slot has a length and a width and extends through a wall of said main body portion, from an outside surface of said main body portion to an inside surface of said main body portion;
wherein, in a closed configuration, said flap closes off said slot; and
wherein, upon rotating said flap away from said main body portion to an open configuration, said slot is exposed.

2. The access port of claim 1, wherein said flap is configured to be stably positioned at any angle relative to a longitudinal axis of said main body portion, between zero degrees and a maximum degree of angulation achievable.

3. The access port of claim 1, wherein said flap is pivotally mounted to said main body portion via a torque and positioning hinge.

4. The access port of claim 1, further comprising an angulation control mechanism configured to be actuated to drive said flap in a controlled manner to open or close said flap position and maintain an angulation of said flap relative to a longitudinal axis of said main body portion at any angle between zero degrees and a maximum angle of the angulation achievable.

5. The access port of claim 4, wherein said angulation control mechanism comprises a ratchet mechanism.

6. The access port of claim 1, further comprising skirts interconnecting sides of said flap with edges of walls of said main body that define said slot.

7. The access port of claim 1, further comprising a connector extending from said first proximal end portion, said connector being configured to facilitate fixing said access port to a stationary object.

8. The access port of claim 1, further comprising a port extending through a wall of said first proximal end portion, said port being configured to allow a portion of a lighting instrument to be inserted therethrough.

9. The access port of claim 8, further comprising a light cable installed through said port.

10. The access port of claim 1, wherein, when in said open configuration, said slot allows an instrument inserted into said main body portion to be angled relative to a longitudinal axis of said main body portion, by passing at least a portion of the instrument through said slot.

11. The access port of claim 1, wherein said slot extends nearly a full length of said main body portion, but is closed off at a distal end of said main body portion where said flap is joined to said main body portion.

12. The access port of claim 1, wherein said first distal end portion of said main body portion is tapered.

13. The access port of claim 12, wherein said second distal end portion of said flap is tapered to conform to said tapered, first distal end portion of said main body portion.

14. The access port of claim 1, further comprising:
at least one longitudinally extending cutout in an inner wall of said main body portion; and
at least one light strip mounted in said at least one longitudinally extending cutout, respectively.

15. The access port of claim 1, provided in the open configuration, in combination with an instrument extending through said access port, wherein a distal end of said instrument extends distally of a distal end of said access port and a shaft of said instrument extends through said slot, to provide increased range of motion of said distal end of said instrument.

16. The access port of claim 1, provided in the closed configuration, in combination with an instrument extending through said access port, wherein a distal end of said instrument extends distally of a distal end of said access port and a shaft of said instrument is prevented from extending through said slot by said flap.

17. The access port of claim 1, wherein said access port is configured and dimensioned for use in minimally invasive spine surgery.

18. The access port of claim 1, further comprising:
a light guide assembly installed in said first proximal end portion;
wherein said light guide assembly comprises a fiber optic cable having optical fibers, said optical fibers being fanned out over an inner circumference of said first proximal end portion to provide even lighting throughout a tubular opening of said tubular main body portion and a surgical target targeted by a distal end opening of said tubular opening.

19. The access port of claim 18, wherein said light guide assembly further comprises a split compression ring, said optical fibers being adhered to said split compression ring and terminating at or proximal of a distal end of said split compression ring.

20. A method of performing minimally invasive surgery comprising:
inserting an access port through the skin of a patient and positioning the access port adjacent or into a surgical target location, wherein the access port includes an elongate, tubular main body portion; a slot extending through the main body portion in a lengthwise direction, wherein the slot extends through a wall of the main body portion, from an outside surface of the main body portion to an inside surface of the main body portion; and a flap pivotally mounted to the main body portion at a distal end portion of the flap, the flap covering the slot in a closed configuration;
inserting an instrument through the access port such that a distal end portion of the instrument extends into the surgical target location;
rotating the flap away from the main body portion to an open configuration to expose the slot; and
manipulating the instrument to pass a shaft portion of the instrument through at least a portion of the slot;
wherein the instrument is manipulatable so as to angle the shaft relative to a longitudinal axis of the access port from a minimal angle of zero degrees to a maximum angle greater than that achievable without passing the shaft portion through at least a portion of the slot, and to any angle therebetween.

21. The method of claim 20, wherein the instrument is a second instrument, said method further comprising:
inserting a first instrument through the access port while in a closed, configuration, prior to the rotating the flap, such that a distal end portion of the first instrument extends into the surgical target location; and
performing work at the surgical target location with the distal end portion of the first instrument while the access port is in the closed configuration.

22. The method of claim 20, further comprising:
removing the instrument from the access port;
rotating the flap to the main body portion to the closed configuration;
inserting a second instrument through the access port, such that a distal end portion of the first instrument extends into the surgical target location; and
performing work at the surgical target location with the distal end portion of the second instrument while the access port is in the closed configuration.

23. The method of claim 20, further comprising:
removing the instrument from the access port;
rotating the flap to the main body portion to the closed configuration; and
removing the access port from the patient while the access port is in the closed configuration.

24. The method of claim 20, further comprising:
fixing at least a portion of the access port to a stationary object.

25. The method of claim 20, wherein the surgical target location is in the spine of the patient.

26. The method of claim 25, wherein the surgical target location is an intervertebral disk space.

27. A method of performing minimally invasive surgery comprising:
inserting an access port in a closed configuration through the skin of a patient and positioning the access port adjacent or into a surgical target location, wherein the access port includes an elongate, tubular main body portion; a slot extending through the main body portion in a lengthwise direction, wherein the slot extends through a wall of the main body portion, from an outside surface of the main body portion to an inside surface of the main body portion; and a flap pivotally mounted to the main body portion at a distal end portion of the flap, the flap covering the slot in the closed configuration;
rotating the flap away from the main body portion to an open configuration to expose the slot, wherein a proximal end of the flap extends out of the patient and is spaced away from the main body portion, and wherein a distal end portion inside the patient remains joined to the main body portion;
performing a procedure through the access port with at least one instrument while the access port is in the open configuration;
rotating the flap to the main body portion to resume the closed configuration; and
removing the access port from the patient while in the closed configuration.

28. The method of claim 27, further comprising performing at least one task through the access port with an instrument while the access port is in the closed configuration.
